(19) United States
(12) Patent Application Publication
(10) Pub. No.: US 2026/0048093 A1
(43) Pub. Date: Feb. 19, 2026

(54) COMBINATION THERAPY OF MIXED HERBAL EXTRACT AND DOCETAXEL FOR PREVENTING OR TREATING CANCER

(71) Applicant: JAEIN R&P INC., Seoul (KR)

(72) Inventors: Seong Gyu KO, Seoul (KR); Yu Jeong CHOI, Seoul (KR); Kangwook LEE, Seoul (KR); Miso JEONG, Suwon-si (KR); Yun-Gyeong KIM, Seoul (KR); Hyun-Ha HWANG, Seoul (KR)

(73) Assignee: JAEIN R&P INC., Seoul (KR)

(21) Appl. No.: 19/100,232

(22) PCT Filed: Aug. 4, 2023
(86) PCT No.: PCT/KR2023/011472

§ 371 Date: Mar. 31, 2025

(30) Foreign Application Priority Data
Aug. 4, 2022 (KR) 10-2022-0097499

(51) Int. Cl. A61K 36/481; A61K 31/337; A61K 36/232; A61K 36/428; A61P 35/00

(52) U.S. Cl. CPC A61K 36/481; A61K 31/337; A61K 36/232; A61K 36/428; A61P 35/00; A61K 2236/333; A61K 2236/51

(57) ABSTRACT

The present invention provides a pharmaceutical composition for preventing or treating triple-negative breast cancer, comprising a mixed extract of Astragalus membranaceus, Angelica gigas, and Trichosanthes kirilowii Maximowicz; and an anticancer agent, and a method for preventing or treating cancer using the pharmaceutical composition.

### TECHNICAL FIELD

The present invention relates to a pharmaceutical composition for preventing or treating cancer, including a mixed extract of Astragalus membranaceus, Angelica gigas, and Trichosanthes kirilowii Maximowicz; and an anticancer agent, and a method for preventing or treating cancer using the pharmaceutical composition.

### BACKGROUND ART

Anticancer agents are a generic term for chemotherapeutic agents other than surgery or radiation therapy among the methods used to treat malignant tumors, and most anticancer agents are drugs that exhibit anticancer activity by inhibiting the synthesis of nucleic acids. For example, compounds such as paclitaxel, docetaxel, etc. are approved by the U.S. Food and Drug Administration and have been used in actual clinical practice, and these compounds provide an effect by excessively stabilizing microtubules of cells to suppress the division of normal cells.

Docetaxel (Taxol), a semisynthetic taxoid produced from the needles of the European yew (Taxus baccata), and its derivative paclitaxel are anticancer agents used to treat solid tumors or highly metastatic malignant tumors, including breast cancer, ovarian cancer, non-small cell lung cancer, and colon cancer, and are among the most important new and active chemotherapeutic agents on the market.

Although docetaxel is an effective anticancer agent, it has limitations such as drug resistance, cancer recurrence, side effects, etc. For example, it is known that the use of high doses of docetaxel induces another level of toxic response, and low to moderate doses of docetaxel do not show significant anti-tumor activity in patients. Therefore, the use of docetaxel as a monotherapy for cancer is currently excluded. Accordingly, there is a need for the development of a combination treatment that can exhibit synergistic anticancer effects when administered in combination with docetaxel.

Meanwhile, Astragalus membranaceus is distributed in South Korea, Japan, Manchuria, northeastern China, and eastern Siberia, as a perennial plant of Wisteria floribunda for. Alba. Astragalus membranaceus is often grown as an herb, and in oriental medicine, a herb which is collected in the fall, removed with outcrops and fine roots, and dried in the sun is called Astragalus membranaceus of medicinal herbs. It is known that Astragalus membranaceus, which is used as it is without removing the bark, has better efficacy. Astragalus membranaceus has effects on robustness, anhidrosis, diuresis, and small boils and is prescribed for physical weakness, fatigue, boredom, cold sweats, etc.

Angelica gigas is a dried root of Angelica gigas which is a perennial grass belonging to Apiaceae, has a sweet taste and a warm nature. Angelica gigas has effects in hematosis, which generates blood when the blood is insufficient, and promotes the blood flow of the coronary artery and activates the generation of erythrocytes. Additionally, it has been reported that an extract of Angelica gigas containing decursin and/or decursinol angelate can be used as an anticancer composition (Korean Patent No. 10-1245328).

Trichosanthes kirilowii Maximowicz refers to a root from which the cortex of perennial Trichosanthes kirilowii or yellow Trichosanthes kirilowii belonging to Cucurbitaceae is removed. It has no small, bitter taste and a cold nature. When resin is damaged due to heat, wasting and thirst disorder, boils, and pus are treated. Trichosanthes kirilowii Maximowicz mainly lowers a fever in the lungs and stomach, generates resin and thereby relieves thirst, and enhances the body.

In this regard, the present inventors have confirmed the anticancer effect of mixed extracts of Astragalus membranaceus, Angelica gigas, and Trichosanthes kirilowii Maximowicz (Korean Patent Publication No. 2014-0145087). However, the synergistic effect of combination treatment using mixed extracts of Astragalus membranaceus, Angelica gigas, and Trichosanthes kirilowii Maximowicz together with existing anticancer agents has not been revealed, and there has been no research thereon.

### DISCLOSURE

### Technical Problem

The present inventors have made extensive efforts to develop a novel anticancer agent that exhibits a synergistic effect in combination treatment with existing anticancer agents, and as a result, have confirmed that a remarkably effective anticancer agent can be obtained by combining herbal extracts of Astragalus membranaceus, Angelica gigas, and Trichosanthes kirilowii Maximowicz with existing anticancer agents, thereby completing the present invention.

### Technical Solution

An object of the present invention is to provide a pharmaceutical composition for preventing or treating cancer, including a mixed extract of Astragalus membranaceus, Angelica gigas, and Trichosanthes kirilowii Maximowicz, wherein the pharmaceutical composition is administered in combination with an anticancer agent, and the anticancer agent is docetaxel or paclitaxel.

In the pharmaceutical composition for preventing or treating cancer of the present invention, in any one specific embodiment, the pharmaceutical composition may reduce the expression level or activity of epidermal growth factor receptor (EGFR).

In the pharmaceutical composition for preventing or treating cancer of the present invention, in any one specific embodiment, the mixed extract is prepared by extracting a mixture of Astragalus membranaceus, Angelica gigas, and Trichosanthes kirilowii Maximowicz with at least one solvent selected from the group consisting of water, alcohols having 1 to 4 carbon atoms, and mixed solvents thereof.

In any one of the specific embodiments described above, the alcohol having 1 to 4 carbon atoms may be 20 to 40% (v/v) ethanol.

In the pharmaceutical composition for preventing or treating cancer of the present invention, in any one specific embodiment, the mixed extract of Astragalus membranaceus, Angelica gigas, and Trichosanthes kirilowii Maximowicz may be mixed in a weight ratio of 0.5 to 5:1:1.

In the pharmaceutical composition for preventing or treating cancer of the present invention, in any one specific embodiment, the cancer is breast cancer, lung cancer, stomach cancer, or prostate cancer.

In any one of the specific embodiments described above, the breast cancer includes triple-negative breast cancer.

In any one of the specific embodiments described above, the lung cancer includes non-small cell lung cancer.

In the pharmaceutical composition for preventing or treating cancer of the present invention, in any one specific embodiment, the composition may further include a pharmaceutically acceptable carrier, excipient or diluent.

In the pharmaceutical composition for preventing or treating cancer of the present invention, the composition may be administered by intraperitoneal administration, intravenous administration, intramuscular administration, subcutaneous administration, intradermal administration, oral administration, topical administration, intranasal administration, intrapulmonary administration, or rectal administration.

Another object of the present invention is to provide a method for preventing or treating cancer, including administering the composition to a subject.

Still another object of the present invention is to provide use of a composition including a mixed extract of Astragalus membranaceus, Angelica gigas, and Trichosanthes kirilowii Maximowicz; and an anticancer agent for the prevention and treatment of cancer.

Still another object of the present invention is to provide use of a mixed extract of Astragalus membranaceus, Angelica gigas, and Trichosanthes kirilowii Maximowicz as an anticancer adjuvant.

Still another object of the present invention is to provide use of a composition including a mixed extract of Astragalus membranaceus, Angelica gigas, and Trichosanthes kirilowii Maximowicz; and an anticancer agent for a combined administration.

Still another object of the present invention is to provide a combined therapeutic agent for a composition including a mixed extract of Astragalus membranaceus, Angelica gigas, and Trichosanthes kirilowii Maximowicz; and an anticancer agent.

Still another object of the present invention is to provide a method for preparing a composition including a mixed extract of Astragalus membranaceus, Angelica gigas, and Trichosanthes kirilowii Maximowicz; and an anticancer agent.

### Advantageous Effects

The mixed extract of Astragalus membranaceus, Angelica gigas, and Trichosanthes kirilowii Maximowicz of the present invention can be used as an excellent anticancer therapeutic agent by administering it in combination with existing anticancer agents and thus exhibiting a synergistic anticancer effect.

### BRIEF DESCRIPTION OF THE DRAWINGS

FIG. 1 is a graph showing the effect of the mixed extract of Astragalus membranaceus, Angelica gigas, and Trichosanthes kirilowii Maximowicz of the present invention or the single administration of docetaxel on the viability of BT-20 and MDA-MB-231 breast cancer cell lines.

FIG. 2 is a graph showing the effect of the mixed extract of Astragalus membranaceus, Angelica gigas, and Trichosanthes kirilowii Maximowicz of the present invention when administered in combination with docetaxel on the viability of BT-20 breast cancer cell line.

FIG. 3 is a graph showing the effect of the mixed extract of Astragalus membranaceus, Angelica gigas, and Trichosanthes kirilowii Maximowicz of the present invention when administered in combination with docetaxel on the viability of MDA-MB-231 breast cancer cell line.

FIG. 4 is a graph showing the effect of the mixed extract of Astragalus membranaceus, Angelica gigas, and Trichosanthes kirilowii Maximowicz of the present invention, and docetaxel when administered alone or in combination on the induction of apoptosis in BT-20 breast cancer cell line.

FIG. 5 is a graph showing the effect of the mixed extract of Astragalus membranaceus, Angelica gigas, and Trichosanthes kirilowii Maximowicz of the present invention, and docetaxel when administered alone or in combination on the inhibition of EGFR phosphorylation in BT-20 and MDA-MB-231 breast cancer cell lines.

FIG. 6 is a graph showing the effect of the mixed extract of Astragalus membranaceus, Angelica gigas, and Trichosanthes kirilowii Maximowicz of the present invention, and docetaxel when administered alone or in combination on the induction of apoptosis in BT-20 breast cancer cell line.

FIG. 7 is a graph showing the effect of the mixed extract of Astragalus membranaceus, Angelica gigas, and Trichosanthes kirilowii Maximowicz of the present invention, and docetaxel when administered alone or in combination on tumor volume in a tumor xenograft mouse model. Tumor size was measured daily, and the blue arrow indicates that DTX was administered intravenously once a week at a dose of 15.28 mg/kg, and the orange arrow indicates that SH003 was administered orally three times a week at a dose of 557.57 mg/kg.

FIG. 8 is a graph showing the effect of the mixed extract of Astragalus membranaceus, Angelica gigas, and Trichosanthes kirilowii Maximowicz of the present invention, and docetaxel when administered alone or in combination on tumor weight and size in a tumor xenograft mouse model.

FIG. 9 is a graph showing the effect of the mixed extract of Astragalus membranaceus, Angelica gigas, and Trichosanthes kirilowii Maximowicz of the present invention, and docetaxel when administered alone or in combination on body weight in a tumor xenograft mouse model.

FIG. 10 is a graph showing the effect of the mixed extract of Astragalus membranaceus, Angelica gigas, and Trichosanthes kirilowii Maximowicz of the present invention, and docetaxel when administered alone or in combination on the viability of H460 lung cancer cell line.

FIG. 11 is a graph showing the effect of the mixed extract of Astragalus membranaceus, Angelica gigas, and Trichosanthes kirilowii Maximowicz of the present invention, and docetaxel when administered alone or in combination on the viability of A549 lung cancer cell line.

FIG. 12 is a graph showing the effect of the Astragalus membranaceus, Angelica gigas, and Trichosanthes kirilowii Maximowicz of the present invention, and docetaxel when administered alone or in combination on the induction of apoptosis in H460 and A549 lung cancer cell lines.

FIG. 13 is a graph showing the effect of the mixed extract of Astragalus membranaceus, Angelica gigas, and Trichosanthes kirilowii Maximowicz of the present invention, and docetaxel when administered alone or in combination on the inhibition of EGFR phosphorylation in H460 and A549 lung cancer cell lines.

FIG. 14 is a graph showing the effects of the mixed extract of Astragalus membranaceus, Angelica gigas, and Trichosanthes kirilowii Maximowicz of the present invention, and docetaxel when administered alone or in combination on (A) tumor size, (B) tumor volume, (C) tumor weight, and (D) body weight in a tumor xenograft mouse model. The tumor size was measured daily, and the blue arrow indicates the administration of docetaxel, and the orange arrow indicates the administration of SH003.

FIG. 15 shows the results of measuring the Combination Index (CI) when the mixed extract of Astragalus membranaceus, Angelica gigas, and Trichosanthes kirilowii Maximowicz (SH003) of the present invention, and docetaxel were administered alone or in combination in prostate cancer cell line DU145.

FIG. 16 shows the results of identifying apoptosis-related proteins by Western blot analysis to compare the efficacy of the mixed extract of Astragalus membranaceus, Angelica gigas, and Trichosanthes kirilowii Maximowicz (SH003) of the present invention and docetaxel when administered alone or in combination in prostate cancer cell line DU145.

FIG. 17 shows that when subcutaneous xenograft was performed for 7 days, the mixed extract of Astragalus membranaceus, Angelica gigas, and Trichosanthes kirilowii Maximowicz (SH003) of the present invention and docetaxel when administered in combination showed (a) relatively more effective tumor growth inhibition than in the single-administration group, (b) no toxicity as no change in the body weight of the mice was observed, and (c) a decrease in cancer cell proliferation by H&E staining. Additionally, it was confirmed that the expression of Ki67, a cell proliferation indicator, was reduced, the expression of Cleaved caspase3, an apoptosis indicator, was increased, the phosphorylation of EGFR was inhibited, and the expression of STAT3 was inhibited. As a result, it was confirmed that apoptosis was induced by inhibiting EGFR-STAT3 signaling (d to e) in a similar manner as compared to the results of in vitro studies.

FIG. 18 shows the results of confirming the cell survival rate when the mixed extract of Astragalus membranaceus, Angelica gigas, and Trichosanthes kirilowii Maximowicz (SH003) of the present invention, and docetaxel were administered alone or in combination to gastric cancer cell line AGS.

FIG. 19 shows the results of confirming whether the mixed extract of Astragalus membranaceus, Angelica gigas, and Trichosanthes kirilowii Maximowicz (SH003) of the present invention and docetaxel when administered alone or in combination induce apoptosis, which is one of the cancer cell suppression mechanisms.

### DETAILED DESCRIPTION OF PREFERRED EMBODIMENTS

The present invention will be described in detail as follows. Meanwhile, each description and embodiment disclosed in this disclosure may also be applied to other descriptions and embodiments. That is, all combinations of various elements disclosed in this disclosure fall within the scope of the present invention. Further, the scope of the present invention is not limited by the specific description described below.

One aspect of the present invention provides a pharmaceutical composition for preventing or treating cancer, including a mixed extract of Astragalus membranaceus, Angelica gigas, and Trichosanthes kirilowii Maximowicz, wherein the pharmaceutical composition is administered in combination with an anticancer agent, and the anticancer agent is docetaxel or paclitaxel.

The mixed extract of Astragalus membranaceus, Angelica gigas, and Trichosanthes kirilowii Maximowicz of the present invention can be effectively used as a therapeutic agent for cancer by administering in combination with existing anticancer agents, thus exhibiting a synergistic anticancer effect.

As used herein, the term “Astragalus membranaceus” refers to a plant with a scientific name of ‘Astragalus membranaceus”; the term “Angelica gigas” refers to a plant with a scientific name of ‘Angelica gigas’, and the term “Trichosanthes kirilowii Maximowicz” refers to a plant with a scientific name of “Trichosanthes kirilowii Maximowicz”.

In the present invention, an ethanol extract of Astragalus membranaceus, Angelica gigas, and Trichosanthes kirilowii Maximowicz may be used interchangeably with “SH003” as having the same meaning.

In the present invention, the Astragalus membranaceus, Angelica gigas, and Trichosanthes kirilowii Maximowicz may be commercially purchased, or may be obtained from collected or cultivated products in nature, but is not limited thereto. Additionally, the extract of Astragalus membranaceus, Angelica gigas, and Trichosanthes kirilowii Maximowicz may be extracted from a natural, hybrid, or variant plant, and may be extracted from a plant tissue culture.

As used herein, the term “extract” includes a liquid extract itself and an extract of any formulation which may be prepared using the extract such as a liquid extract obtained from a mixture of Astragalus membranaceus, Angelica gigas, and Trichosanthes kirilowii Maximowicz by extraction treatment, a diluent or concentrate of the liquid extract, a dried product obtained by drying the liquid extract, a crude or purified product of the liquid extract, or a mixture thereof, etc.

In the pharmaceutical composition for preventing or treating cancer of the present invention, in any one specific embodiment, the mixture of Astragalus membranaceus, Angelica gigas, and Trichosanthes kirilowii Maximowicz may be extracted with one or more solvents selected from the group consisting of water, alcohols having 1 to 4 carbon atoms, and mixed solvents thereof, specifically, an ethanol extract, but is not limited thereto.

In any one of the specific embodiments described above, the alcohol having 1 to 4 carbon atoms may be 20 to 40% (v/v) ethanol, specifically, 30% (v/v) ethanol, but is not limited thereto.

In the mixed extract of Astragalus membranaceus, Angelica gigas, and Trichosanthes kirilowii Maximowicz, a method of extracting the mixture is not particularly limited, and it may be extracted according to a method commonly used in the art. Non-limiting examples of the extraction method may include hot water extraction, ultrasonic extraction, filtration, reflux extraction, etc., and these may be carried out alone or in a combination of two or more methods.

Additionally, the extract may be prepared in the form of a dry powder after extraction to be used, but is not limited thereto.

In the pharmaceutical composition for preventing or treating cancer of the present invention, in any one specific embodiment, the mixed extract of Astragalus membranaceus, Angelica gigas, and Trichosanthes kirilowii Maximowicz may be mixed in a weight ratio of 0.5 to 5:1:1, specifically, 1 to 3:1:1, and more specifically, 1:1:1, but is not limited thereto.

As used herein, the term “anticancer agent” generically refers to a well-known drug used for the treatment of cancer cells, which acts on a variety of metabolic pathways of cancer cells and exhibits cytotoxicity or cytostatic effects in cancer cells, and includes all anticancer agents developed to date, including antimetabolites, plant alkaloids, topoisomerase inhibitors, alkylating agents, anticancer antibiotics, hormone preparations and other drugs.

The anticancer agents which are contained in the pharmaceutical composition of the present invention and particularly exhibit synergistic effects with the mixed extract of Astragalus membranaceus, Angelica gigas, and Trichosanthes kirilowii Maximowicz may be cisplatin drugs, such as cisplatin, carboplatin and heptaplatin, gemcitabine, 5-fluorouracil, and Taxane drugs such as docetaxel and a derivative thereof, paclitaxel, preferably, docetaxel and paclitaxel, and more preferably, docetaxel, but are not limited thereto. These anticancer agents may be prepared by a known method or may be commercially purchased.

Docetaxel (Taxol), a semi-synthetic taxoid produced from the needles of the European yew (Taxus baccata), and its derivative paclitaxel are anticancer agents used to treat solid tumors or highly metastatic malignant tumors, and are among the most important new and active chemotherapeutic agents on the market.

Although docetaxel is an effective anticancer agent, it has limitations such as drug resistance, cancer recurrence, side effects, etc. For example, it is known that a high dose of docetaxel induces another level of toxicity, and low or medium doses of docetaxel do not show significant antitumor activity in patients. Thus, the use of docetaxel as a single treatment for cancer is currently excluded. Accordingly, the development of a combination therapy that can show synergistic anticancer effects when administered in combination with docetaxel is required

As used herein, the term “cancer” refers to abnormally grown lumps attributable to the autonomous overgrowth of body tissues and is also referred to as a tumor. Specifically, the term may include breast cancer, lung cancer, stomach cancer, or prostate cancer.

Cancer is a disease caused by abnormal lumps of uncontrolled cell growth in the tissues of the body, and is the result of uncontrolled cell growth in a variety of tissues.

As used herein, the term “breast cancer” is a cancer in which abnormal tissue continues to grow in the breast or spreads to other organs, and specifically, it may be triple-negative breast cancer.

As used herein, the term “triple-negative breast cancer” refers to breast cancer representing about 15% of all breast cancers, and is generally known as breast cancer that relapses faster than other types of breast cancers and has a relatively low survival rate. Triple-negative breast cancer does not have receptors for three types of hormones, i.e., estrogen receptor, progesterone, and human epidermal growth factor receptor 2 (HER2), and thus does not respond well to antihormone therapy and targeted therapy, and has a poor prognosis.

As used herein, the term “lung cancer” is a cancer that occurs in the lung itself or metastasizes to the lungs from another organ, and in the present invention, the lung cancer may be non-small cell lung cancer.

As used herein, the term “non small lung cancer” may be used interchangeably with non-small cell lung cancer. Non-small cell lung cancer is a type of carcinoma, which refers to all epithelial lung cancers that are not small lung cancer and accounts for approximately 85% to 90% of total lung cancers. Symptoms of non-small cell lung cancer include persistent cough, chest pain, weight loss, nail damage, joint pain, shortness of breath, etc. Because non-small cell lung cancer usually progresses slowly, producing no symptoms in the early stage, early recognition and treatment are difficult, and it is likely to be diagnosed after the cancer has spread to the whole-body including bones, liver, small intestines, brain, etc.

As used herein, the term “stomach cancer” is a general term for cancers that occur in the stomach. The most common type of stomach cancer is adenocarcinoma, but in addition, rare types such as lymphoma that occurs in the lymphatic tissue of the stomach, gastrointestinal tumor that occurs in the stromal cells of the stomach, sarcoma that originates from non-epithelial tissues, and neuroendocrine tumor that secretes hormones may also occur.

As used herein, the “prostatic carcinoma” refers to a cancer that occurs in the prostate, most of which is adenocarcinoma occurring in the prostate glands. These types of cancer may be classified depending on the degree of differentiation of the tumor tissue and the characteristics of the cells. A widely used grading system was proposed by a pathologist named Donald Gleason, and is divided into grades from 1, the best, to 5, the worst. It is reported that cancers with higher differentiation have a better prognosis.

In the present invention, the cancer may be any cancer whose symptoms may be alleviated, reduced, improved, or treated by the pharmaceutical composition for preventing or treating, including the mixed extract of Astragalus membranaceus, Angelica gigas, and Trichosanthes kirilowii Maximowicz provided in the present invention; and an anticancer agent, but is not particularly limited thereto.

As used herein, the term “prevention” means all of the actions by which the occurrence of cancer is restrained or delayed by administering the pharmaceutical composition for preventing or treating, including the mixed extract of Astragalus membranaceus, Angelica gigas, and Trichosanthes kirilowii Maximowicz; and an anticancer agent.

As used herein, the term “treatment” means all of the actions by which symptoms in an individual who is suspected of having cancer and has developed cancer have taken a turn for the better or been modified favorably by administering the pharmaceutical composition.

As used herein, the term “combined administration” means concurrent or sequential treatment.

In one specific embodiment of the present invention, it was confirmed that when the mixed extract of Astragalus membranaceus, Angelica gigas, and Trichosanthes kirilowii Maximowicz; and docetaxel were administered in combination, a synergistic anticancer effect was obtained, and in particular, it was confirmed that the therapeutic effect was significantly increased upon combined administration.

In the pharmaceutical composition for preventing or treating cancer, in any one embodiment, the pharmaceutical composition may reduce the expression level or activity of epidermal growth factor receptor (EGFR), but is not limited thereto.

The epidermal growth factor receptor (EGFR) has been identified as a target for the treatment of a number of cancers, in particular, solid tumors, as it is involved in regulating cellular functions important in the proliferation and survival of cancer cells. Increased expression of EGFR has been observed in bladder, breast, glioblastoma, head and neck, lung and stomach cancers.

In the present invention, the pharmaceutical composition may exhibit a synergistic effect in anticancer treatment by reducing the expression level or activity of epidermal growth factor receptor (EGFR).

In the pharmaceutical composition for preventing or treating cancer, in any one embodiment, the pharmaceutical composition may further include a pharmaceutically acceptable carrier, excipient, or diluent, but is not limited thereto.

The pharmaceutical composition may further include a pharmaceutically acceptable carrier, excipient, or diluent commonly used in the preparation of the pharmaceutical composition. The carrier may include a non-naturally occurring carrier.

As used herein, the term “pharmaceutically acceptable” means no toxicity to cells or humans exposed to the composition.

Specifically, the pharmaceutical composition may be formulated into oral preparations such as powders, granules, tablets, capsules, suspensions, emulsions, syrups, aerosols, etc., and preparations for external application, suppository, or sterile injectable solution according to common methods. In the present invention, the carrier, excipient, and diluent which may be included in the pharmaceutical composition may include lactose, dextrose, sucrose, sorbitol, mannitol, xylitol, erythritol, maltitol, starch, acacia rubber, alginate, gelatin, calcium phosphate, calcium silicate, cellulose, methyl cellulose, microcrystalline cellulose, polyvinyl pyrrolidone, water, methylhydroxybenzoate, propylhydroxybenzoate, talc, magnesium stearate, and mineral oil. When the composition is formulated, the formulation may be prepared by using a diluent or an excipient, such as a filler, an extender, a binder, a wetting agent, a disintegrating agent, and a surfactant which are generally used. Solid formulations for oral administration may include tablets, pills, powders, granules, capsules, etc., and these solid formulations may be prepared by mixing with at least one excipient, for example, starch, calcium carbonate, sucrose, lactose, gelatin, etc. In addition to such simple excipients, lubricants such as magnesium stearate or talc may also be used. Liquid formulations for oral administration may include suspensions, solutions for internal use, emulsions, syrups, etc., and may include various excipients, such as wetting agents, flavors, fragrances, preservatives, etc., in addition to water and liquid paraffin, which are commonly used as a simple diluent. Formulations for parenteral administration may include sterile aqueous solutions, non-aqueous solvents, suspensions, emulsions, lyophilized preparations, or suppositories. The non-aqueous solutions and the suspensions may include propylene glycol, polyethylene glycol, vegetable oil such as olive oil, injectable ester such as ethyl oleate, etc. The base for suppositories may include witepsol, macrogol, tween 61, cacao butter, laurin butter, glycerogelatin, etc.

In the pharmaceutical composition for preventing or treating cancer of the present invention, in any one embodiment, the composition may be administered by intraperitoneal administration, intravenous administration, intramuscular administration, subcutaneous administration, intradermal administration, oral administration, topical administration, intranasal administration, intrapulmonary administration, or rectal administration, but the administration route is not limited thereto.

Another aspect of the present invention provides a method for preventing or treating cancer, including administering the composition to a subject.

In particular, the cancer, prevention, and treatment are as defined above.

As used herein, the term “administration” means the introduction of a predetermined substance into a subject by any suitable method.

As used herein, the term “subject” means all animals of rats, mice, livestock, etc., including humans, who have developed or are at risk of developing cancer. In one specific embodiment, it may be mammals including humans, but the subject is not limited thereto.

The method for preventing or treating cancer of the present invention may specifically include administering a pharmaceutical composition for preventing or treating cancer, which includes a mixed extract of Astragalus membranaceus, Angelica gigas, and Trichosanthes kirilowii Maximowicz; and an anticancer agent, in a pharmaceutically effective amount.

As used herein, the term “pharmaceutically effective amount” refers to an amount sufficient to treat a disease at a reasonable benefit/risk ratio applicable to medical treatment and not cause side effects, and the level of the effective amount may be readily determined by those skilled in the art, depending on a variety of factors including the patient's sex, age, body weight, health condition, the kind of disease, severity, activity of the drug, sensitivity to the drug, administration method, administration time, administration route, excretion rate, duration of treatment, drugs used in combination or concurrently, and other factors known in the medical field.

Specifically, the compound of the present invention may be administered at a daily dose of 0.0001 to 100 mg/body weight (kg), and more preferably, 0.001 to 100 mg/body weight (kg) based on the solid content. Administration may be performed with the above recommended administration dose once a day or several times a day.

In the method for preventing or treating cancer of the present invention, the administration route and mode for administering the composition are not particularly limited, and the composition may be administered according to any administration route and mode, as long as it is able to reach a desired site. Specifically, the composition may be administered via a variety of routes including oral or parenteral routes, and non-limiting examples of the administration route may include an oral, rectal, topical, intravenous, intraperitoneal, intramuscular, intra-arterial, transdermal, intranasal route, or inhalation.

### MODE FOR CARRYING OUT THE INVENTION

Hereinafter, the present invention will be described in more detail through exemplary embodiments to help in the understanding of the present invention. However, these exemplary embodiments may be modified in many different forms and the scope of the present invention should not be construed as being limited to the embodiments set forth below. The exemplary embodiments of the present invention are provided for the purpose of a full-depth explanation to those who have an average knowledge in the art.

### Example 1. Preparation of Fraction of Mixed Herbal Ethanol Extract (SH003) and Preparation of Docetaxel

Astragalus membranaceus (Am), Angelica gigas (Ag), and Trichosanthes kirilowii Maximowicz (Tk) were mixed in a weight ratio (w/w) of 1:1:1, respectively, and the mixture was added to an extractor, and extracted with 30% (v/v) ethanol for 3 hours at 100° C. The extract was filtered and the filtrate was concentrated under reduced pressure and then dried to obtain a mixed extract of Astragalus membranaceus, Angelica gigas, and Trichosanthes kirilowii Maximowicz. The dried extract was dissolved in 30% ethanol and stored at −80° C. until use.

Docetaxel (DTX, Sigma-Aldrich, St. Louis, MO, USA) was commercially purchased and then dissolved in DMSO and stored at −20° C.

### Experimental Example 1. Analysis of Synergistic Effect by Combined Administration of Mixed Extract of the Present Invention and Docetaxel in Triple-Negative Breast Cancer

### Experimental Example 1-1. Analysis of Synergistic Effect by Combined Administration of Mixed Extract of the Present Invention and Docetaxel on Viability of Triple-Negative Breast Cancer Cells

In order to confirm a synergistic effect of the mixed extract prepared in Example 1 and docetaxel on the viability of the triple-negative breast cancer cells, an MTT assay was performed according to a conventionally known method. As cancer cell lines, BT-20 (TNBC, triple-negative breast cancer, non-invasive) and MDA-MB-231 (TNBC, highly metastatic) were used.

In more detail, in the MDA-MB-231 triple-negative breast cancer cell line incubated on a 96-well plate, the mixed extract of Astragalus membranaceus, Angelica gigas, and Trichosanthes kirilowii Maximowicz, which was prepared in Example 1 (SH003, 100, 300 and 500 μg/mL), and docetaxel (DTX, 1, 10, 100 and 1000 nM) were treated with various concentrations and then incubated for 24 hours under a condition of 37° C. and 5% CO₂. Thereafter, a 3-(4,5-Dimethylthiazol-2-yl)-2,5-diphenyltetrazolium bromide (MTT) reagent was treated in each cell, the cells were further incubated for 4 hours, and then a supernatant was removed and 100 μL of dimethyl sulfoxide (DMSO) was added. Finally, absorbance was measured at a wavelength of 590 nm and the absorbance value was compared with a control group which was not treated with any material.

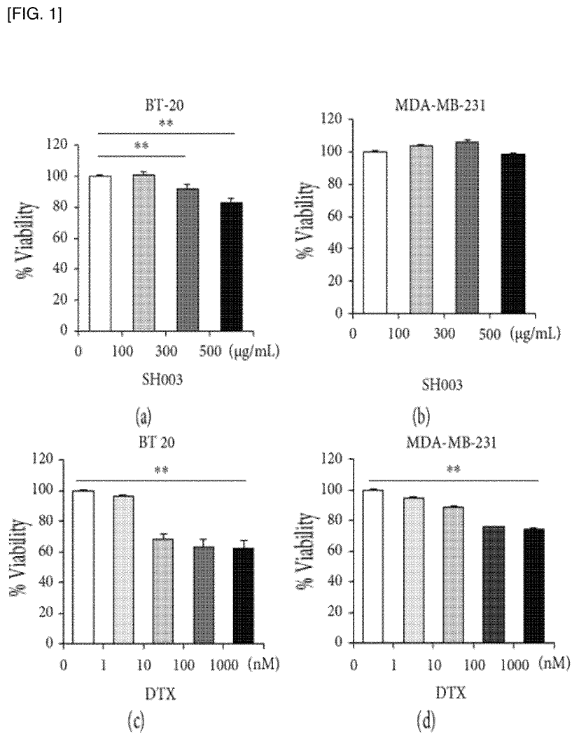

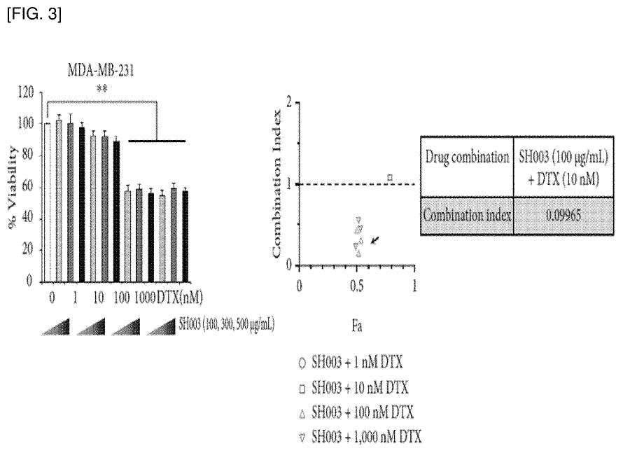

The results were illustrated in FIGS. 1 and 3.

As illustrated in FIGS. 1(a) and 1(b), it was confirmed that SH003 had no effect on the viability of BT-20 and MDA-MB-231 cells, and as illustrated in FIGS. 1(c) and 1(d), DTX decreased the viability of both BT-20 and MDA-MB-231 cells in a dose-dependent manner.

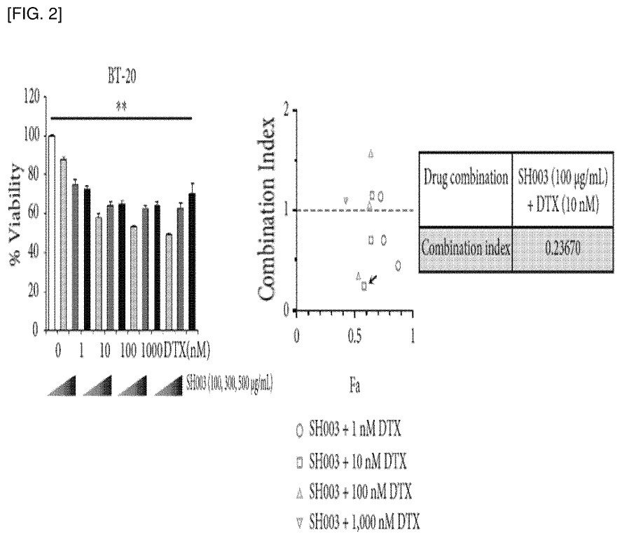

Meanwhile, as illustrated in FIGS. 2 and 3, it was confirmed that when DTX and SH003 were administered in combination, the viability of triple-negative breast cancer cells was significantly inhibited.

Subsequently, in order to confirm whether SH003 had a synergistic effect with DTX, the combination index (CI) of the drug pair was calculated using CompuSyn software, where CI<1, CI=1, and CI>1 indicate a synergistic effect, an additive effect, and an antagonistic effect, respectively. Fa indicates the inhibition rate. The results are illustrated in Tables 1 and 2 below.

TABLE 1
Combination Index of SH003 and DTX in BT-20 cells

| | BT-20 |
|---|---|
| Drugs | Combination |

| DTX(nM) | SH003(μg/mL) | Index (CI) |
|---|---|---|
| 1 | 100 | 0.44311 |
| | 300 | 0.70508 |
| | 500 | 1.14198 |
| 10 | 100 | 0.23670 |
| | 300 | 0.70086 |
| | 500 | 1.14927 |
| 100 | 100 | 0.35019 |
| | 300 | 1.05284 |
| | 500 | 1.57479 |
| 1000 | 100 | 1.12051 |
| | 300 | 4.74933 |
| | 500 | 10.8015 |

TABLE 2
Combination Index of SH003 and DTX in MDA-MB-231 cells

| | MDA-MB-231 |
|---|---|
| Drugs | Combination |

| DTX(nM) | SH003(μg/mL) | Index (CI) |
|---|---|---|
| 1 | 100 | 1.85E20 |
| | 300 | 5.90E23 |
| | 500 | 7.01930 |
| 10 | 100 | 3.82112 |
| | 300 | 3.16236 |
| | 500 | 1.17233 |
| 100 | 100 | 0.09965 |
| | 300 | 0.27457 |
| | 500 | 0.42340 |
| 1000 | 100 | 0.19020 |
| | 300 | 0.45114 |
| | 500 | 0.57037 |

As illustrated in Tables 1 and 2 above, the highest synergistic effects in BT-20 and MDA-MB-231 cells were observed in the combination of 100 μg/ml of SH003 and 10 nM of DTX (CI: 0.24) and the combination of 100 μg/mL of SH003 and 100 nM of DTX (CI: 0.1), respectively. These results suggest that SH003 and DTX have a synergistic effect by blocking excessive toxic accumulation at relatively low concentrations of DTX.

### Experimental Example 1-2. Confirmation of Effect of Inducing Apoptosis in Triple-Negative Breast Cancer Cells by Combined Administration of Mixed Extract of the Present Invention and Docetaxel

In order to detect apoptosis, BT-20 and MDA-MB-231 cells were treated with SH003 and/or DTX for 24 hours, and then apoptosis was analyzed by flow cytometry.

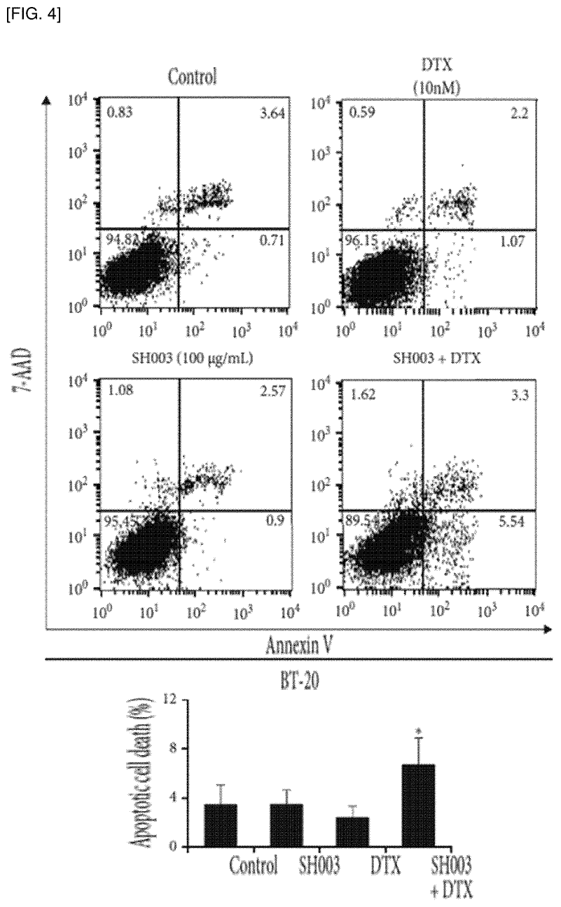

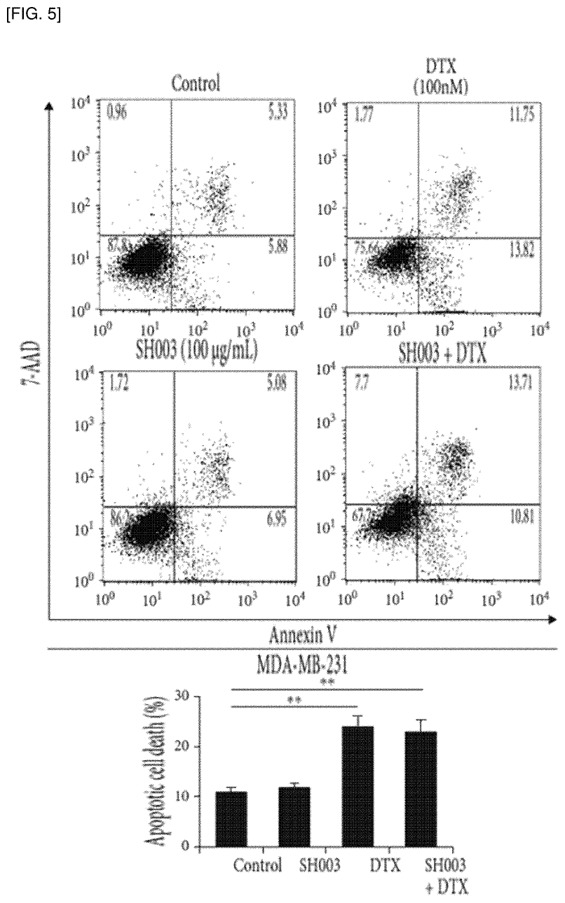

Specifically, it was analyzed by Annexin V/7-AAD double staining. More specifically, the cells were stained with Annexin V and then stained with 7-AAD in the dark for 15 minutes at room temperature, and then the stained cells were detected by FACSCalibur (BD Biosciences, San Jose, CA, USA), and the apoptotic cells were analyzed using CellQuest Pro version 5.2 (BD Biosciences, San Jose, CA, USA) software. The results are illustrated in FIGS. 4 and 5.

As illustrated in FIGS. 4 and 5, when SH003 and DTX were administered in combination, it was confirmed that the combined administration in BT-20 cells improved the level of apoptotic cells compared to the single administration of SH003 or DTX, or in a similar manner to the single administration of DTX.

These results suggest that the combined administration of the mixed extract of the present invention and docetaxel increases apoptosis, thereby increasing the sensitivity of SH003 or DTX to the triple-negative breast cancer cells.

### Experimental Example 1-3. Confirmation of Effect of Inducing Apoptosis Through Inhibition of EGFR Signaling Pathway in Triple-Negative Breast Cancer Cells by Combined Administration of Mixed Extract of the Present Invention and Docetaxel

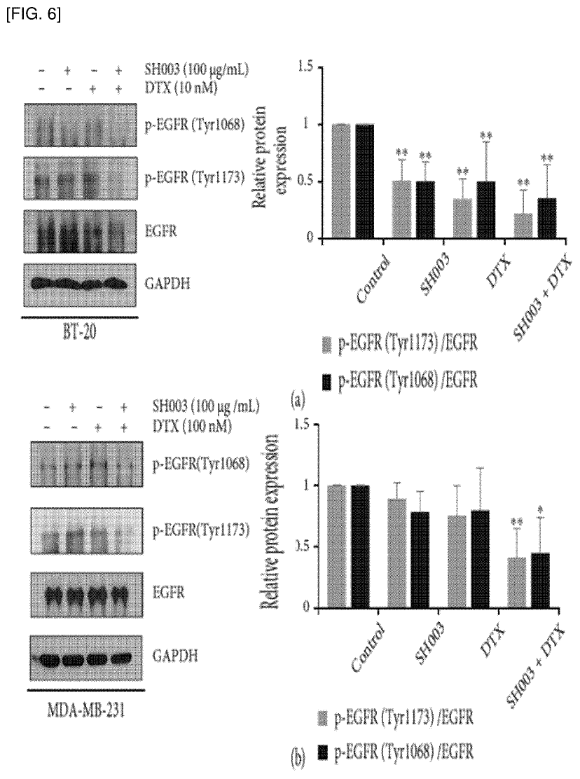

In order to confirm whether the EGFR signaling pathway is inhibited by the combined administration of the mixed extract of the present invention and docetaxel, a Western blot analysis was performed. The results are illustrated in FIG. 6.

As illustrated in FIGS. 6(a) and 6(b), it was confirmed that the combined administration of SH003 and DTX inhibited the phosphorylation of EGFR in both BT-20 and MDA-MB-231 cells.

### Experimental Example 1-4. Confirmation of Effect of Inhibiting the Growth of Triple-Negative Breast Cancer Tumors and EGFR Phosphorylation by Combined Administration of Mixed Extract of the Present Invention and Docetaxel in Animal Model

The effect of the combined administration of the mixed extract of the present invention and docetaxel was investigated in an animal model. All animal studies were approved by Kyung Hee University-Institutional Animal Care & Use Committee (KHU-IACUC). Five-week-old female BALB/c nude mice were purchased from Nara Biotech (Seoul, Republic of Korea). The mice were allowed free access to food and water and were housed in appropriate isolation cages at room temperature (22-25° C.) under a 12-h light/12-h dark cycle in the absence of pathogens. In order to establish a tumor xenograft mouse model, BT-20 cell suspension (1×10⁷ cells) in 100 μL PBS was subcutaneously inoculated into the right flank of the mice. The mice were divided into four groups: control group (n=3), SH003 (n=4), DTX (n=4), and SH003+DTX (n=5). When the tumor volume reached 100 mm³, the drug was injected. DTX was administered intravenously via the tail vein once a week at 15.277 mg/kg, while DMSO was administered to the control or SH003 single-administration group. SH003 was administered orally at 557.569 mg/kg three times a week, while saline was administered to the control or DTX single-administration group. The body weight was measured three times a week, and tumor volume was analyzed daily for 17 days.

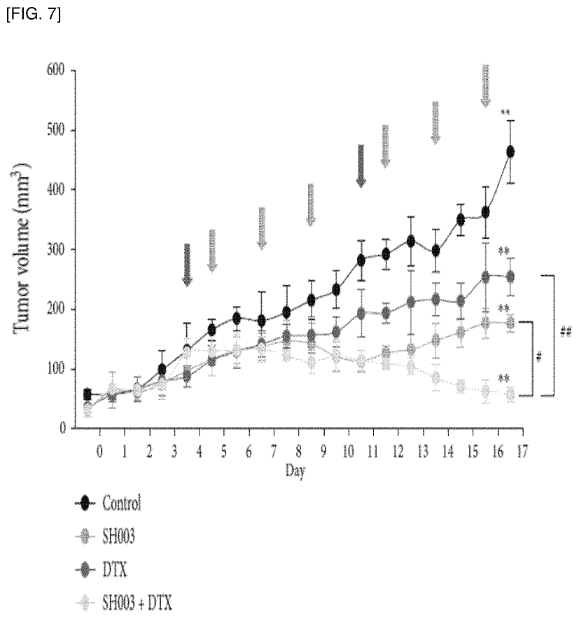

Subsequently, the mice were euthanized and the tumors were isolated. The tumor tissues were fixed and mounted on slide glass for hematoxylin and eosin (H&E) staining, and the tissues were incubated with H&E solution. The H&E images were obtained with a microscope (Carl Zeiss, Germany) at 40× magnification. The results are illustrated in FIGS. 7 to 9.

As illustrated in FIG. 7, it was confirmed that the combined administration of SH003 and DTX most effectively inhibited tumor growth compared to each of the single administration groups.

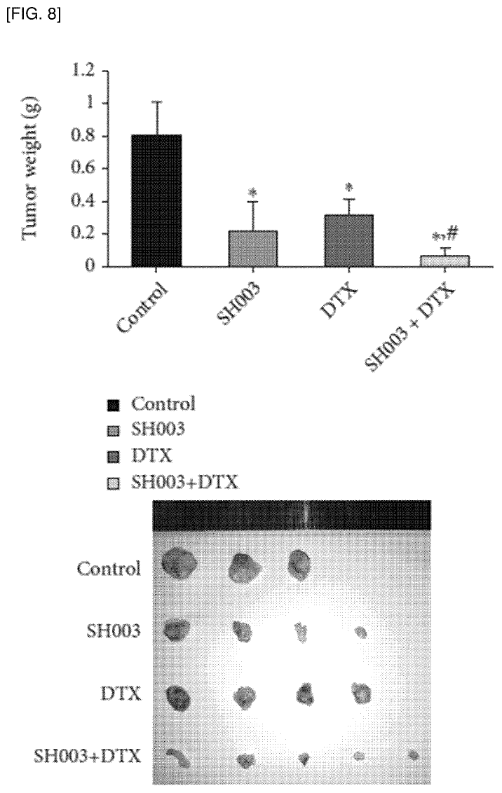

In addition, as illustrated in FIG. 8, it was confirmed that the combined administration of SH003 and DTX significantly reduced the weight and size of the tumor compared to each of the single-administration groups.

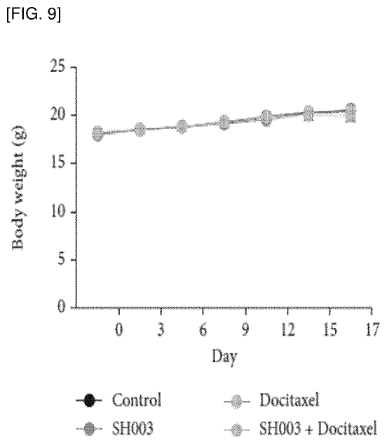

Meanwhile, as illustrated in FIG. 9, it was confirmed that the single administrations of SH003 and DTX and combined administration of SH003 and DTX did not have an effect on body weight.

In summary, it was confirmed that the mixed extract of Astragalus membranaceus, Angelica gigas, and Trichosanthes kirilowii Maximowicz of the present invention induces apoptosis of triple-negative breast cancer cells in vitro and in vivo without causing side effects and enhances the anticancer effect of docetaxel by inhibiting the EGFR signaling pathway.

### Experimental Example 2. Analysis of Synergistic Effect by Combined Administration of Mixed Extract of the Present Invention and Docetaxel in Non-Small Cell Lung Cancer

### Experimental Example 2-1. Analysis of Synergistic Effect by Combined Administration of Mixed Extract of the Present Invention and Docetaxel on Viability of Non-Small Cell Lung Cancer Cells

In order to confirm the synergistic effect of the mixed extract prepared in Example 1 and docetaxel on the viability of non-small cell lung cancer cells, cell viability was measured using the WST colorimetric assay. As cancer cell lines, H460 (no. 30177, Korean Cell Line Bank, Seoul, Korea) and A549 (no. 10185, Korean Cell Line Bank, Seoul, Korea), which are non-small cell lung cancer cell lines, were used.

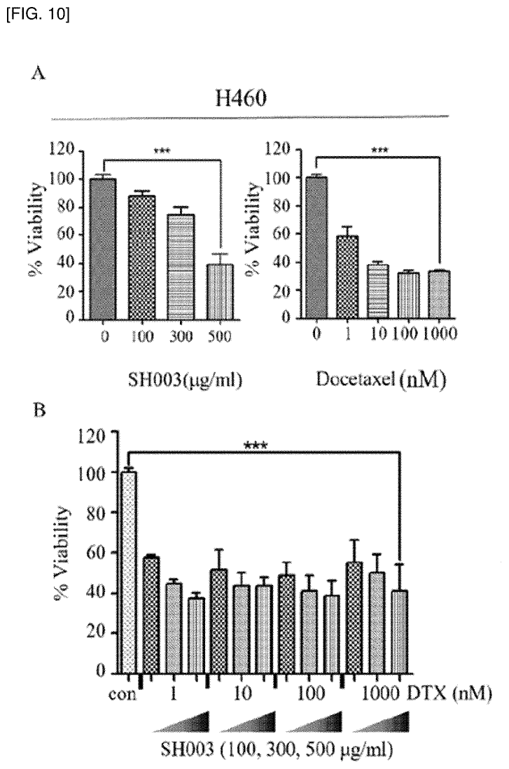

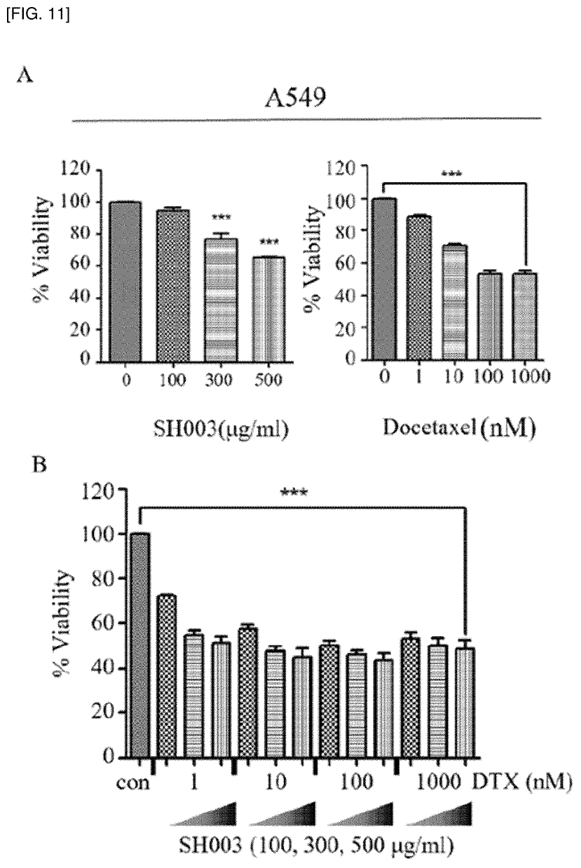

Specifically, a total of 10⁵ cells per well were seeded in a 6-well plate before treatment. After 24 hours, the cells were treated with SH003 (100, 300, and 500 μg/mL), DTX (1, 10, 100, and 1000 nM), or a combination thereof for 24, 48, and 72 hours. The control medium contained 0.15% ethanol and 0.1% DMSO, which had no cytotoxic effect. After treatment, the cells were incubated with WST for 1 hour, and then the absorbance was measured at 450 nm using an ELISA reader (Molecular Devices, Palo Alto, CA, USA). The results are illustrated in FIGS. 10 and 11.

As illustrated in FIG. 10A, both SH003 and DTX single administrations inhibited H460 cell growth in a dose-dependent manner, and as illustrated in FIG. 10B, when DTX and SH003 were administered in combination, it was confirmed that the growth of H460 cells was inhibited more effectively compared to the single administrations.

As illustrated in FIG. 11A, both SH003 and DTX single administrations inhibited A549 cell growth in a dose-dependent manner, and as illustrated in FIG. 11B, when DTX and SH003 were administered in combination, it was confirmed that the growth of A549 cells was inhibited more effectively compared to the single administrations.

Subsequently, in order to confirm whether SH003 has a synergistic effect with DTX, the combination index (CI) of the drug pair was calculated using CompuSyn software, where CI<1, CI=1, and CI>1 indicate a synergistic effect, an additive effect, and an antagonistic effect, respectively. Fa indicates the inhibition rate. The results are illustrated in Table 3 below.

TABLE 3
Combination Index of Combination Treatment
of SH003 and Docetaxel in H460 cells H460

| SH003(μg/mL) | Docetaxel (nM) | Effect | CI Value |
|---|---|---|---|
| 100 | 1 | 0.577 | 4.54621 |
| 300 | 1 | 0.44 | 0.69068 |
| 500 | 1 | 0.372 | 0.77952 |
| 100 | 10 | 0.516 | 7.92777 |
| 300 | 10 | 0.434 | 1.3307 |
| 500 | 10 | 0.437 | 1.77591 |
| 100 | 100 | 0.485 | 32.6988 |
| 300 | 100 | 0.413 | 4.79899 |
| 500 | 100 | 0.388 | 2.87891 |
| 100 | 1000 | 0.553 | 2165.1 |
| 300 | 1000 | 0.5 | 493.736 |
| 500 | 1000 | 0.413 | 43.6589 |

As illustrated in Table 3 above, the combination of 300 μg/mL of SH003 and 1 nM of DTX showed the lowest CI (CI=0.69) in H460 cells.

### Experimental Example 2-2. Confirmation of Effect of Inducing Apoptosis in Non-Small Cell Lung Cancer Cells by Combined Administration of Mixed Extract of the Present Invention and Docetaxel

In order to investigate the effect of increasing apoptosis by combined administration of the mixed extract of the present invention and docetaxel, Annexin V/7-AAD double staining was used for analysis.

Specifically, H460 and A549 cells were stained with Annexin V and then stained with 7-AAD in the dark for 15 minutes at room temperature, and then the stained cells were detected by FACSCalibur (BD Biosciences, San Jose, CA, USA), and the apoptotic cells were analyzed using CellQuest Pro version 5.2 (BD Biosciences, San Jose, CA, USA) software. The results are illustrated in FIG. 3.

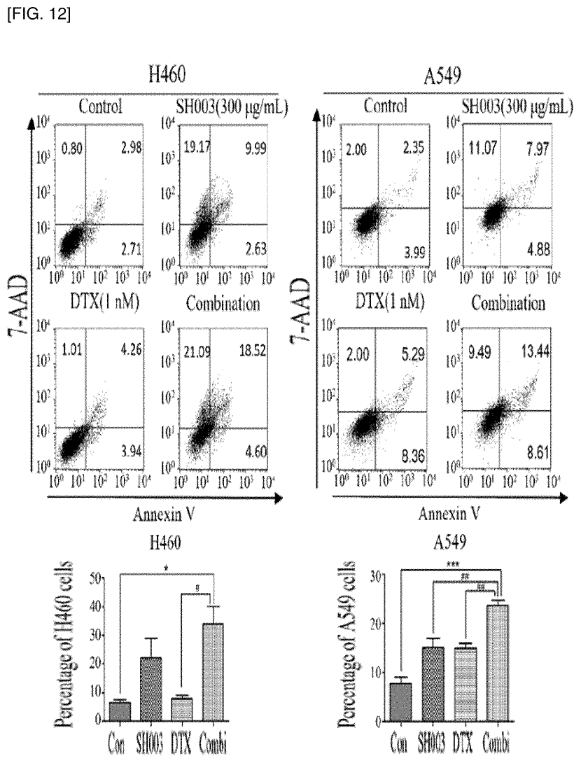

As illustrated in FIG. 12, the treatment with DTX for 24 hours did not significantly affect H460 and A549 cells compared to the control group, whereas SH003 and combined administration induced apoptosis. Specifically, it was confirmed that the combined administration significantly increased apoptosis in H460 (34.07%) and A549 cells (23.74%).

These results suggest that the combined administration of the mixed extract of the present invention and docetaxel increases apoptosis, thereby increasing the sensitivity of SH003 or DTX to the non-small cell lung cancer cells.

### Experimental Example 2-3. Confirmation of Effect of Inducing Apoptosis Through Inhibition of EGFR Signaling Pathway in Non-Small Cell Lung Cancer Cells by Combined Administration of Mixed Extract of the Present Invention and Docetaxel

In order to confirm whether the EGFR signaling pathway is inhibited by the combined administration of the mixed extract of the present invention and docetaxel, a Western blot analysis was performed. The results are illustrated in FIG. 4.

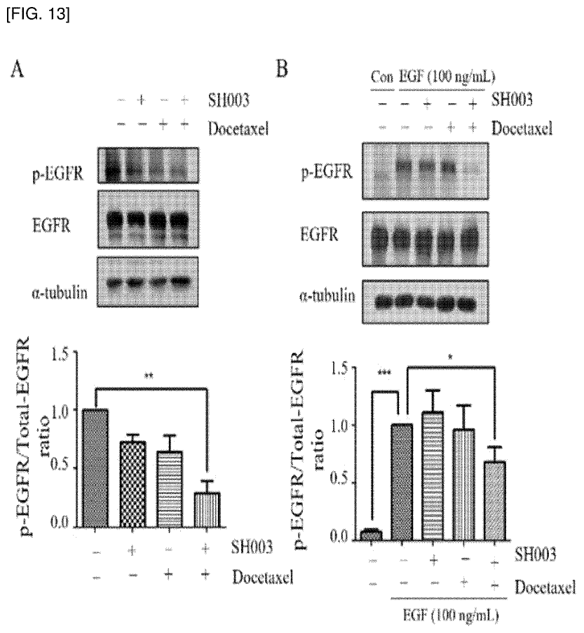

As illustrated in FIG. 13A, the combined administration of SH003 and DTX synergistically inhibited the expression of p-EGFR (Y1068) and phosphorylation of EGFR in H460 cells. In addition, as illustrated in FIG. 13B, the combined administration of SH003 and DTX significantly reduced the p-EGFR level, meanwhile the EGF treatment increased the p-EGFR level.

### Experimental Example 2-4. Confirmation of Effect of Inhibiting Tumor Growth and EGFR Phosphorylation by Combined Administration of Mixed Extract of the Present Invention and Docetaxel in Animal Model

The effect of the combined administration of the mixed extract of the present invention and docetaxel was investigated in an animal model. All procedures of animal experimentation, including maintenance and euthanasia, were approved by Kyung Hee University Institutional Animal Care and Use Committee (KHU-IACUC-KHSASP-21-211). Five-week-old male Balb/c nude mice were purchased from Nara Biotech (Seoul, Republic of Korea) and acclimated to a 12-h light/dark cycle at 22-23° C. and 40-60% humidity. The mice were divided into four groups: control (n=3), SH003 administration (n=4), DTX administration (n=4), and combined administration (n=5). In order to establish a tumor xenograft mouse model, H460 cell suspension (1×10⁷ cells) in 100 μL PBS was subcutaneously inoculated into the right flank of the mice. Thereafter, 200 μL of SH003 (557.569 mg/kg) was administered orally three times a week, and 50 μL DTX (15.277 mg/kg) was administered intravenously via the tail vein once a week for 3 weeks. Saline and DMSO were used as vehicles. The body weight was measured three times a week, and tumor volume was analyzed daily. On day 21, the mice were sacrificed using carbon dioxide and cervical dislocation was performed. Tumor tissues were isolated after euthanasia. The tumor tissues were fixed and mounted on slide glass for hematoxylin and eosin (H&E) staining, and the tissues were incubated with H&E solution. The H&E images were obtained with a microscope (Carl Zeiss, Germany) at 40× magnification. The results are illustrated in FIG. 5

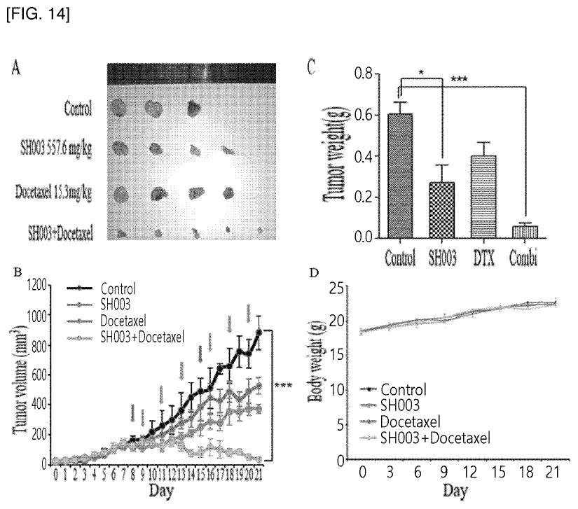

As illustrated in FIGS. 14A to 14C, it was confirmed that the combined administration of SH003 and DTX significantly reduced the tumor volume and weight compared to each of the single administration groups.

Meanwhile, as illustrated in FIG. 14D, it was confirmed that the single administrations of SH003 and DTX and the combined administration thereof did not have an effect on body weight. This suggests that combined administration of the mixed extract of the present invention and docetaxel synergistically inhibits tumor growth without causing toxicity in mice.

In summary, it was confirmed that the mixed extract of Astragalus membranaceus, Angelica gigas, and Trichosanthes kirilowii Maximowicz of the present invention induces apoptosis of non-small cell lung cancer cells in vitro and in vivo without causing side effects and enhances the anticancer effect of docetaxel by inhibiting the EGFR signaling pathway.

### Experimental Example 3. Analysis of Synergistic Effect of Combined Administration of Mixed Extract of the Present Invention and Docetaxel in Prostate Cancer and Stomach Cancer

The synergistic effect of the combined administration of the mixed extract of the present invention and docetaxel was confirmed in stomach cancer and prostate cancer using a similar experimental method to Experimental Examples 1 and 2 above.

### Experimental Example 3-1. Analysis of Synergistic Effect by Combined Administration of Mixed Extract of the Present Invention and Docetaxel in Prostate Cancer

First, in order to verify the efficacy of the combined effect of the mixed extract SH003 of the present invention and docetaxel in the prostate cancer cell line DU145, an MTT assay was performed for single treatment and combined treatment. The Combination Index (CI) was measured based on the results of the combined treatment.

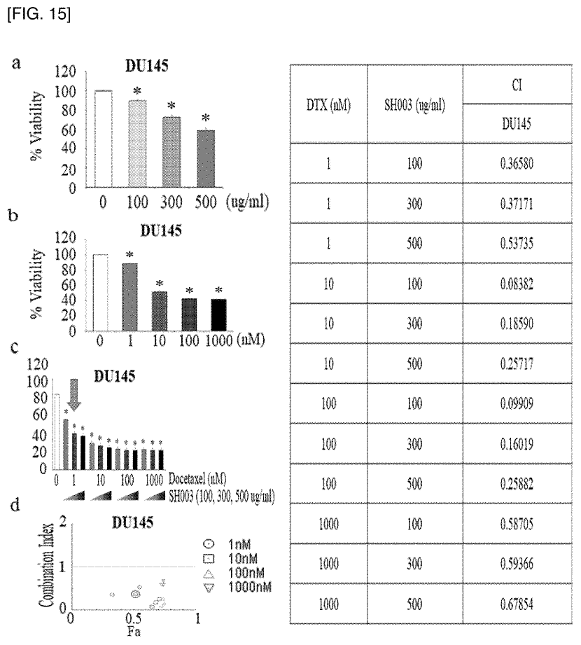

As a result, as can be seen in FIG. 15, it was confirmed that the combination of 1 nM of docetaxel and 300 of μg/mL SH003 showed an excellent synergistic effect. Herein, if CI is less than 1, it can be interpreted as having a synergistic effect, if CI is 1, it can be interpreted as having a cooperative effect, and if CI is greater than 1, it can be interpreted as having an antagonistic effect.

Based on these results, the concentration showing a cell viability close to 50% was selected and used for the subsequent experiments.

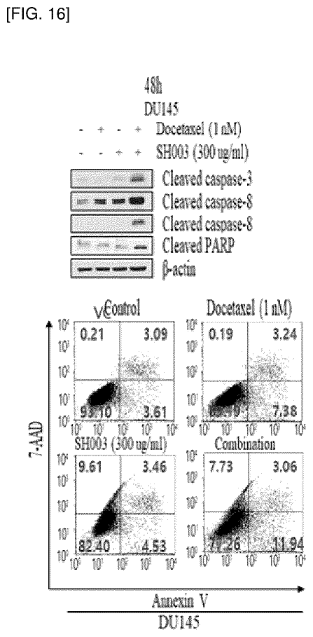

In order to compare the efficacy of the combined effect of SH003 and docetaxel in the prostate cancer cell line DU145, apoptosis-related proteins were verified through Western blot analysis. In particular, apoptosis was most clearly confirmed by the combination of concentrations obtained through the MTT assay. A relatively larger number of cells undergoing apoptosis were also confirmed through flow cytometry analysis (FIG. 16).

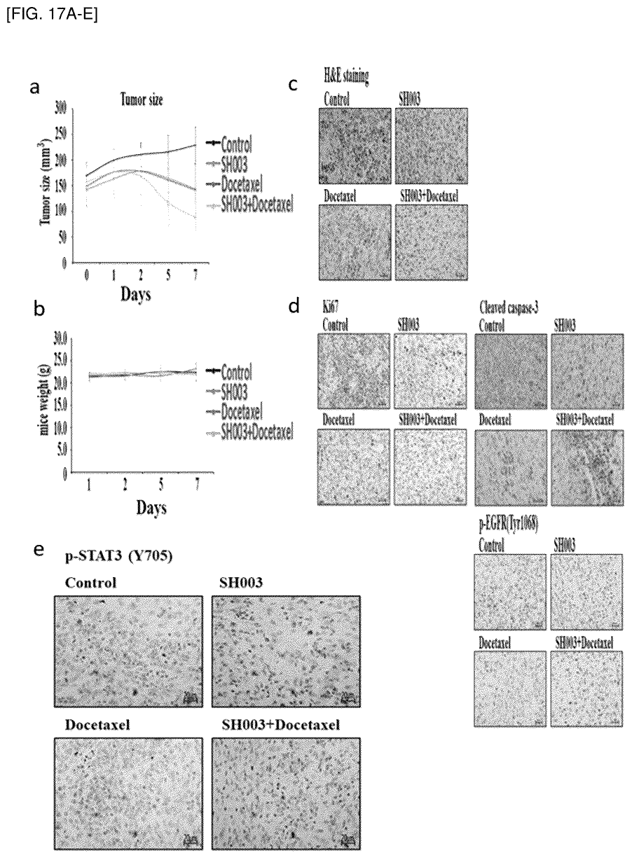

Finally, a) when a subcutaneous xenograft was performed for 7 days, the group that received combination therapy showed relatively more effective tumor growth inhibition than in the other groups. b) Since there was no change in body weight of the mice, it was confirmed that there was no toxicity. c) It was confirmed that cancer cell proliferation was reduced through H&E staining. d) to g) The expression of Ki67, a cell proliferation indicator, also decreased. It was confirmed that the expression of cleaved caspase3, an apoptosis indicator, increased, the phosphorylation of EGFR was inhibited, and the expression of STAT3 was also inhibited. As a result, it was confirmed that apoptosis was induced by inhibiting EGFR-STAT3 signaling in the same manner compared to the results of the in vitro study (FIG. 17).

### Experimental Example 3-2. Analysis of Synergistic Effect by Combined Administration of Mixed Extract of the Present Invention and Docetaxel in Stomach Cancer

It was confirmed whether the mixed extract (SH003) of the present invention has a synergistic effect by combined administration in gastric cancer using a gastric cancer cell line.

First, the cell survival rate by combined treatment was confirmed. For reference, the specific experimental method is the same as the method of Experimental Examples 1 and 2.

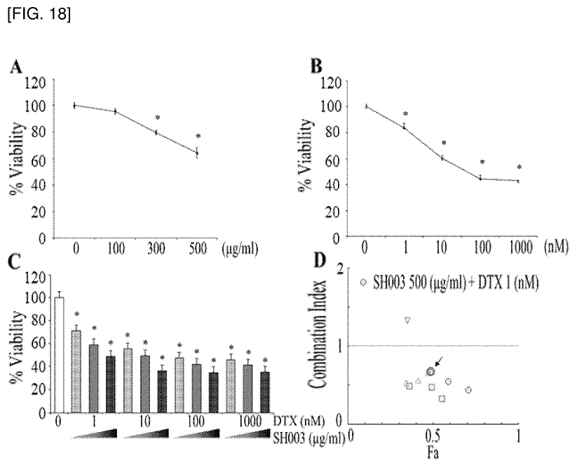

As a result, as can be confirmed in FIG. 18, it was confirmed that the AGS cell survival rate was decreased in a concentration-dependent manner when the gastric cancer cell line AGS was treated with SH003 and docetaxel in combination and each was administered as single administration. In particular, as a result of confirming the cell survival rate effect by combined treatment, it was confirmed that the cell survival rate was significantly reduced when SH003 and docetaxel were treated in combination as compared to when treated as single administration.

Furthermore, the combination index (CI) was calculated to determine whether the decrease in cell viability due to combination treatment had a synergistic effect compared to single treatment. As a result, the CI value was found to be less than 1, clearly confirming that there was a synergistic effect. This shows that there is a synergistic effect due to the combination treatment of the mixed extract of the present invention and docetaxel.

Based on these results, additional experiments were conducted at a combination treatment concentration with a low CI value.

Subsequently, the effect of inducing apoptosis by combined treatment of the mixed extract of the present invention and docetaxel was confirmed. In particular, it was intended to confirm whether the combined treatment induced apoptosis, which is one of the cancer cell inhibition mechanisms.

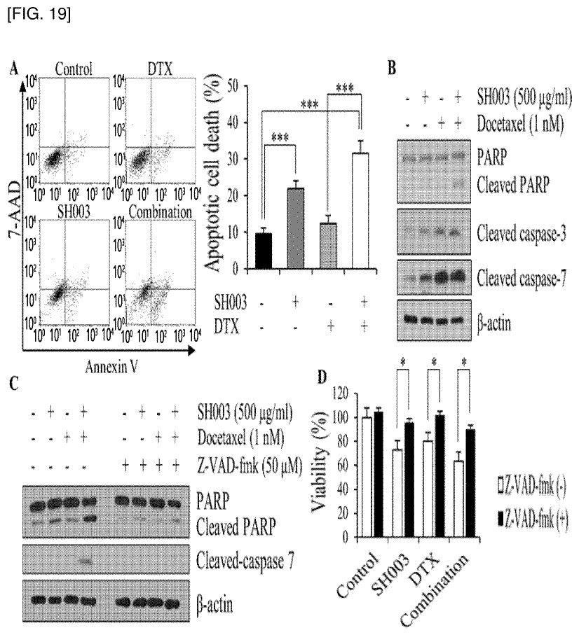

As a result, as can be confirmed in FIG. 19, the result of confirming the effect of inducing apoptosis through Annexin V and 7-AAD staining confirmed that apoptosis increased when SH003 and Docetaxel were treated in combination as compared to when treated as single administration. Additionally, as a result of confirming whether the expression of PARP, cleaved caspase-3, and cleaved caspase-7, which are protein markers related to the cell death mechanism, was regulated, it was confirmed that all proteins related to cell death increased by combined treatment.

In addition, in order to confirm whether the apoptosis-induced mechanism by combined treatment is the main mechanism of gastric cancer cell inhibition, the change in cell viability was confirmed by treatment with a caspase inhibitor, an apoptosis-related protein. As a result, it was confirmed that the expression of PARP and cleaved-caspase 7, which were increased by combined treatment, was reduced when treated with a caspase inhibitor, and the cell viability inhibition effect induced by combined treatment was inhibited.

In conclusion, it was confirmed that the effect of inducing apoptosis by combined treatment of the mixed extract of the present invention and docetaxel was the main inhibition mechanism of AGS gastric cancer cell line inhibition.

In summary, it was confirmed that the combined administration of the mixed extract of the present invention and the anticancer agent had a significantly superior cancer treatment effect compared to when each was administered as single administration, and thus it was confirmed that the mixed extract of the present invention significantly improved the anticancer effect of anticancer agents, especially docetaxel, without causing side effects.

Based on the above description, it will be understood by those skilled in the art that the present invention may be implemented in a different specific form without changing the technical spirit or essential characteristics thereof. In this regard, it should be understood that the above embodiment is not limitative, but illustrative in all aspects. The scope of the present invention is defined by the appended claims rather than by the description preceding them, and therefore all changes and modifications that fall within the metes and bounds of the claims, or equivalents of such metes and bounds are therefore intended to be embraced by the claims.

1. A method for preventing or treating cancer, comprising administering a composition, which includes a mixed extract of Astragalus membranaceus, Angelica gigas, and Trichosanthes kirilowii Maximowicz,
wherein the composition is administered in combination with an anticancer agent,
wherein the anticancer agent is a taxane drug.

2. The method of claim 1, wherein the composition reduces the expression level or activity of epidermal growth factor receptor (EGFR).

3. The method of claim 1, wherein the mixed extract is prepared by extracting a mixture of Astragalus membranaceus, Angelica gigas, and Trichosanthes kirilowii Maximowicz with at least one solvent selected from the group consisting of water, alcohols having 1 to 4 carbon atoms, and mixed solvents thereof.

4. The method of claim 2, wherein the alcohol having 1 to 4 carbon atoms is 20 to 40% (v/v) ethanol.

5. The method of claim 1, wherein the mixed extract of Astragalus membranaceus, Angelica gigas, and Trichosanthes kirilowii Maximowicz is mixed in a weight ratio of 0.5 to 5:1:1.

6. The method of claim 1, wherein the cancer is breast cancer, lung cancer, stomach cancer, or prostate cancer.

7. The method of claim 6, wherein the breast cancer is triple-negative breast cancer.

8. The method of claim 6, wherein the lung cancer is non-small cell lung cancer.

9. The method of claim 1, further comprising a pharmaceutically acceptable carrier, excipient or diluent.

10. The method of claim 1, wherein the composition is administered by intraperitoneal administration, intravenous administration, intramuscular administration, subcutaneous administration, intradermal administration, oral administration, topical administration, intranasal administration, intrapulmonary administration, or rectal administration.

11. The method of claim 1, wherein the taxane drug is selected from the group comprising docetaxel, paclitaxel and combination thereof.
